(19) United States
(12) Patent Application Publication
(10) Pub. No.: US 2010/0284928 A1
(43) Pub. Date: Nov. 11, 2010

(54) METHODS FOR IN VIVO ATHEROSCLEROTIC PLAQUE CHARACTERIZATION USING MAGNETIC SUSCEPTIBILITY TO IDENTIFY SYMPTOM-PRODUCING PLAQUES

(75) Inventors: Subha V. Raman, Columbus, OH (US); Orlando P. Simonetti, Columbus, OH (US); Jay L. Zweier, Blacklick, OH (US)

Correspondence Address: MACMILLAN SOBANSKI & TODD, LLC, ONE MARITIME PLAZA FIFTH FLOOR, 720 WATER STREET, TOLEDO, OH, 43604-1619, US

(73) Assignee: THE OHIO STATE UNIVERSITY RESEARCH FOUNDATION, Columbus, OH (US)

(21) Appl. No.: 12/811,292

(22) PCT Filed: Jan. 2, 2009
(86) PCT No.: PCT/US2009/030011

§ 371 Date: Jul. 30, 2010

Related U.S. Application Data
(60) Provisional application No. 61/010,120, filed on Jan. 4, 2008.

(51) Int. Cl. A61K 49/06; A61K 49/00; A61B 5/055; G06K 9/00

(52) U.S. Cl. 424/9.3; 424/9.1; 600/411; 382/131

(57) ABSTRACT

Methods and uses of in vivo atherosclerotic plaque characterization in a subject in need thereof, that include detecting a level of one or more iron complexes in plaque in the subject, and comparing detected level to a known value determined from subjects without an atherosclerotic condition are disclosed.

### CROSS-REFERENCE TO RELATED APPLICATIONS

This application claims the benefit of U.S. Provisional Application No. 61/010,120 filed Jan. 4, 2008, the entire disclosure of which is expressly incorporated herein by reference.

### STATEMENT REGARDING FEDERALLY SPONSORED RESEARCH

This invention was made with Government support and the Government has rights in this invention under the grant under the National Institutes of Health NHLBI R21 HL080474-01.

### TECHNICAL FIELD AND INDUSTRIAL APPLICABILITY OF THE INVENTION

This invention is directed to certain methods for the characterization of in vivo atherosclerotic plaque using magnetic susceptibility, methods for distinguishing symptom-producing plaques, and methods using such characterization in the diagnosis and treatment of plaque-related disorders. By using imaging techniques and data to differentiate between symptom-producing and clinically-silent plaque, symptom-producing plaque can be identified and removed from the subject.

### BACKGROUND OF THE INVENTION

Atherosclerosis is a major cause of cardiovascular disease, including acute coronary syndromes and ischemic strokes. With increasing recognition that the plaque microenvironment determines clinical sequelae rather than degree of vessel stenosis alone, better strategies to characterize plaque are needed to improve prevention and treatment. Since it was first proposed that relative iron depletion was protective against cardiovascular disease, the quest to demonstrate iron's role in atherosclerosis has focused on its ability to catalyze the peroxidation of low-density lipoprotein (LDL).

Microhemorrhage into atherosclerotic plaque with macrophage-mediated phagocytosis and degradation of aged red blood cells leads to accumulation of redox-active iron. Via Fenton chemistry, iron catalyzes the generation of oxidized LDL. See FIG. 1. Oxidized LDL, but not native LDL, binds the macrophage scavenger-receptor, leading to unregulated uptake, foam cell formation, and accelerated atherogenesis.

Despite these established pathophysiologic mechanisms, studies relating to iron and atherosclerosis have provided conflicting results. Iron has consistently been found in greater concentrations in atherosclerotic plaque compared with normal arterial tissue. In animal models, iron overload accelerates atherogenesis. Epidemiologic studies, however, have yielded equivocal results when comparing serologic markers of total body iron stores with the incidence of atherosclerotic disease.

Notably, little work has involved direct in vivo examination of plaque iron, particularly with an appreciation of the different species of iron in biologic tissues. Free or low molecular weight iron exists as Fe(II) and Fe(III) cations. Iron may be incorporated into hemoglobin or bound to the storage proteins ferritin and hemosiderin, both of which cause measurable changes in local magnetic field homogeneity. This change can be appreciated qualitatively using magnetic resonance T2*-weighted imaging or quantified using the relaxation parameter T2*. T2* quantification allows for the accurate estimation of tissue iron content. It is to be understood that the time constant known as T2* is a transverse relaxation time constant in nuclear magnetic resonance and magnetic resonance imaging, which is dependent on static local magnetic field inhomogeneity, as well as dynamic spin-spin interactions.

TABLE 1
Multiple in vivo and ex vivo techniques exist
to measure these various forms of iron

| Method | Type of Iron Detected |
|---|---|
| Inductively-coupled plasma mass | Total iron |
| spectroscopy | |
| Electron paramagnetic resonance | Low molecular weight |
| | Fe(III) |
| T2* magnetic resonance imaging | Iron aggregates |

Inductively coupled plasma mass spectroscopy (ICP-MS) is used to measure total iron content. Electron paramagnetic resonance (EPR) is sensitive to several forms of iron, iron storage, and iron transport proteins; the g≈4 peak is specific for Fe(III) with rhombic coordination symmetry. Electron paramagnetic resonance does not detect the reduced state of iron, Fe(II). T2*-weighted magnetic resonance imaging (MRI) is sensitive to iron clusters as occurs in ferritin- or hemosiderin-bound iron, but, until the present invention, a quantitative estimation of T2* has not been used to understand iron's role in the microenvironment of human atherosclerotic plaque.

There is a need, therefore, to develop methods for the characterization of atherosclerotic plaque and for methods that can be used to diagnose and treat disorders that are affected by atherosclerotic plaque.

There is a further need for a method and a system to distinguish between “harmful or symptom-producing” coronary atherosclerotic plaques from “benign or clinically-silent” plaques. It is to be understood that the term “harmful or symptom-producing plaque” can generally include the presence of plaque that produces one or more atherosclerotic symptoms in a subject, as compared to “benign or asymptomatic plaque” which can generally include plaque in an asymptomatic subject.

There is a further need to provide such a method which is minimally invasive, and/or which is useful to allow a practitioner to identify and/or treat one or more affected sites of such “harmful or symptom-producing” plaque.

Various objects and advantages of this invention will become apparent to those skilled in the art from the following detailed description of the preferred embodiment, when read in light of the accompanying drawings.

### BRIEF DESCRIPTION OF THE FIGURE(S)

FIG. 1: LDL Peroxidation Catalyzed by Iron. The Haber-Weiss reaction and Fenton chemistry use iron in generating free radicals that oxidize low-density lipoprotein (LDL). Microhemorrhage into atherosclerotic plaque with macrophage-mediated phagocytosis and degradation of aged red blood cells leads to accumulation of redox-active iron. Oxidized LDL binds the macrophage scavenger-receptor, leading to unregulated uptake, foam cell formation, and accelerated atherogenesis.

FIGS. 2A-2D: T2*-Weighted Imaging and Intraplaque T2* Measurement. Serial T2*-weighted dark blood images at various echo times (FIG. 2A, echo time [TE] 2.7 ms; FIG. 2B, TE 7.6 ms; FIG. 2C, TE 12.5 ms; FIG. 2D, TE 17.4 ms) obtained at the location of maximum stenosis allow drawing of a region of interest (FIG. 2D) on all the images encompassing the plaque for measurement of mean T2* within the plaque.

FIG. 2E: T2* is measured in a given plaque by fitting the measured signal intensities at each TE to an exponential decay curve e−^{TE/T2*}. ECA=external carotid artery; ICA=internal carotid artery; J=internal jugular vein; VA=vertebral artery.

FIGS. 3A-3B: Intraplaque T2* by Symptom Status.

FIG. 3A: Plot of in vivo magnetic resonance-derived T2* values of carotid artery plaque in asymptomatic versus symptomatic patients shows shorter T2* times in symptomatic patients. Mean±SD is shown.

FIG. 3B: Bland-Altman analysis shows good agreement of carotid T2* measurement between independent observers (r=0.88).

FIGS. 4A-4C: EPR Spectroscopy Detection of Iron in Carotid Plaques. Electron paramagnetic resonance (EPR) is a powerful and minimally invasive technique to identify and quantify the presence of paramagnetic ferric iron [Fe(III)] within an explanted carotid specimen. The EPR spectra were recorded on frozen tissue at 77 K. Representative EPR spectra from control carotid artery (FIG. 4A), asymptomatic patient's carotid plaque (FIG. 4B), and symptomatic patient's carotid plaque (FIG. 4C) are shown. Atherosclerotic plaque samples demonstrate the high-spin rhombic iron species peak at a magnetic field of ˜1,500 Gauss, corresponding to a g value of 4.3. Although no rhombic iron signal was observed in control carotid tissue, a prominent signal was present in asymptomatic patients' carotid plaques; however, in symptomatic patients' plaques, the level of this signal was significantly decreased.

FIGS. 5A-5C: Plaque Histopathology.

FIG. 5A: Histopathological section with Prussian blue staining at low and high (inset) magnifications demonstrates iron deposits in plaque.

FIG. 5B: Staining for glycophorin A shows evidence of red blood cell membrane fragments within plaque.

FIG. 5C: Staining for Factor VIII demonstrates neovascularization in the plaque neointima.

FIG. 6: Model of Mechanism. The Examples herein show similar total iron in symptom-producing versus non-symptom-producing carotid plaques; however, the former group had less paramagnetic iron by EPR and greater T2*-shortening iron species. This difference shows a shift from paramagnetic Fe(III) to iron aggregates that have a greater effect on local magnetic susceptibility, measurable using the tissue-specific magnetic resonance imaging relaxation time T2*.

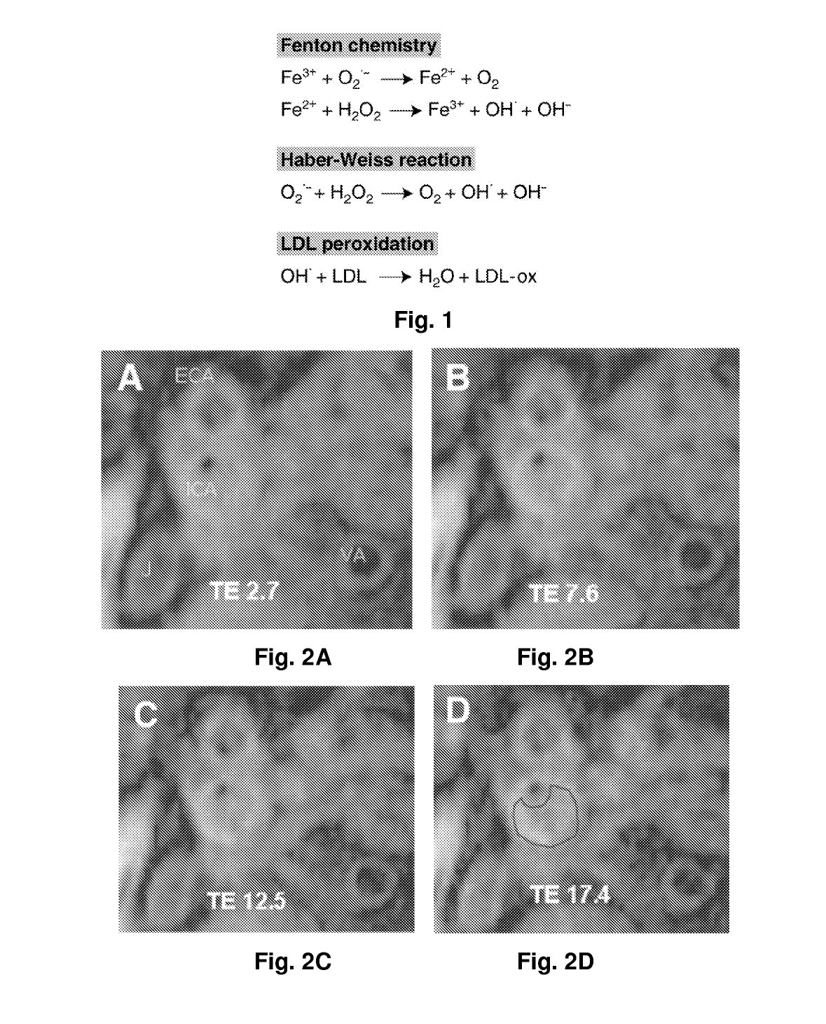

### DETAILED DESCRIPTION OF PREFERRED EMBODIMENT(S)

In a broad aspect, and without limiting the scope of the invention, there is provided herein a novel method for assessing the risk that an individual is likely to have an acute cardiovascular event such as a heart attack or stroke. Heart attacks and strokes are an ultimate and acute manifestation of underlying cardiovascular disease which is largely due to atherosclerotic processes. Unfortunately, sudden cardiac death is often the first sign of the cardiovascular disease. In other circumstances, the patient may be found to have buildups of plaque in their arteries. While some individuals may not suffer an acute event even though there is a presence of plaque, other individuals are at high risk for a cardiovascular event. The unpredictable nature of heart attack and stroke, and the need for cost-effective risk stratification in large groups of asymptomatic at-risk populations are unsolved problems in cardiovascular healthcare.

Magnetic resonance imaging (“MRI”) is a technology that is useful for the detection and assessment of many pathological and physiological alterations in living tissue. An MRI scan of a patient is non-invasive and harmless to such patient. An MRI scan generally utilizes magnetic and radio frequency fields to elicit a response from a given patient's tissue and to provide high quality image “slices” (two-dimensional image reconstructions of a two-dimensional cross-section of the patient's body). An MRI scan generally uses a “T1 relaxation time” and a “T2 relaxation time” to distinguish between tissue types. The T1 relaxation time (also called spin lattice or longitudinal relaxation time) and the T2 relaxation time (also called spin relaxation time or transverse relaxation time) are biological parameters used in MRIs to distinguish between tissue types. The T1 relaxation time is a measure of the time taken to realign with an external magnetic field. The T1 constant may indicate how quickly the spinning nuclei will emit their absorbed radio frequencies into the surrounding tissue. The T2 relaxation time is dependent on the exchanging of energy with nearby nuclei. T2 is generally defined as the decay of the magnetization perpendicular to the main magnetic field (in an ideal homogenous field). The term “T1-weighted imaging” can be used to describe an image where most of the contrast between tissues is due to differences in the T1 value. The term “T2-weighted imaging” can be used to create an image with an image contrast that generally relies upon local dephasing of spins caused by random spin-spin interaction following the application of the transverse energy pulse. T2* is a third relaxation time that can be used to characterize tissue. T2* incorporates T2 relaxation mechanisms, as well as local static magnetic field inhomogeneities that will contribute to more rapid decay of transverse magnetization. “T2*-weighted imaging” can be used to create an image with an image contrast that generally relies upon local dephasing of spins caused by local magnetic field inhomogeneities following the application of the transverse energy pulse.

In a first broad aspect, there is provided herein a method for an in vivo characterization of atherosclerotic plaque characterization. In certain embodiments, the method includes detecting a level of one or more iron complexes in plaque in the subject, and comparing detected levels to a known value determined from subjects without an atherosclerotic condition.

In another broad aspect, there is provided herein a noninvasive method for in vivo atherosclerotic plaque characterization that includes measuring carotid plaque using a T2*-weighted imaging measurement to distinguish symptom-producing plaque from clinically-silent plaque in a subject.

In certain embodiments, the method includes distinguishing the magnetic susceptibility of symptom-producing plaque as compared to clinically-silent plaque in the subject.

In certain embodiments, the iron complex comprises paramagnetic-Fe(III) complexes.

In certain embodiments, the presence of decreased levels of paramagnetic-Fe(III) complexes is indicative of symptom-producing plaque.

In certain embodiments, greater concentrations of one or more iron complexes are present in atherosclerotic plaque compared with normal arterial tissue.

In certain embodiments, a change in iron concentration is qualitatively measured using magnetic resonance T2*-weighted imaging. In certain embodiments, the change in iron concentration is quantified using a T2* relaxation parameter.

In certain embodiments, the T2* quantification allows for a substantially accurate estimation of tissue iron content in the plaque.

In certain embodiments, the method includes MRI imaging of the plaque in vivo.

In another broad aspect, there is provided herein a method for detecting an atherosclerotic condition in a subject, comprising: a) acquiring MRI data from the subject with a magnetic resonance imaging system that measures T2* in plaque present in the subject; and b) comparing the MRI data of step a) to one or more known parameters; and c) indicating a difference between the data of step a) with the known parameters.

In another broad aspect, there is provided herein a method for plaque characterization in a subject in need thereof, comprising: obtaining at least a first set of image data created in response to an MRI scan of at least a portion of plaque in the subject, wherein the MRI scan includes T2*-weighted imaging; and determining the presence of at least one iron complex present in the plaque; wherein the presence of the at least one iron complex at an altered level is indicative of symptom-producing plaque in the subject.

In certain embodiments, the subject being scanned was injected with a contrast agent.

In another broad aspect, there is provided herein a system for plaque characterization, comprising: an imaging system generating at least a first set of image data of plaque in a subject in response to a first magnetic energy level; the first set of image data corresponding to a T2*-weighted imaging of the plaque, and a processing device in communication with the imaging system obtaining at least the first set of image data from the imaging system and displaying an indication of one or more iron complexes present in the plaque.

In certain embodiments, the imaging system comprises a magnetic resonance imaging system.

In certain embodiments, the imaging system is remotely located from the processing device.

In certain embodiments, the processing device is in communication with the imaging system over a network.

In another broad aspect, there is provided herein a computer program product for plaque characterization in cardiac applications, the product comprising: a storage medium readable by a processing circuit and storing instructions for executing a method for plaque characterization by the processing circuit, the method comprising a method as described in any of the preceding claims.

In another broad aspect, there is provided herein a method to improve risk assessment for an acute cardiovascular event in a patient presenting with plaque, comprising detecting a level of one or more iron complexes in plaque in the subject, and comparing the detected level to a known value determined from subjects without an atherosclerotic condition.

In certain embodiments, the level of the iron complex is detected with a magnetic resonance imaging scan.

In another broad aspect, there is provided herein a method for characterizing response to therapy in a clinical trial of a medication, device and/or drug comprising determining a level of one or more iron complexes in a trial participant.

In another broad aspect, there is provided herein an imaging system for differentiating between symptom-producing plaque and clinically-silent plaque tissue in a subject, comprising: a) at least one imaging device for providing imaging data; b) a processor receiving the imaging data and executing software to evaluate the image data according to at least one algorithm; and c) the algorithm processing the imaging data and outputting information relating to at least a level of one or more iron complexes present in the plaque tissue.

In certain embodiments, the imaging system further includes, for guided removal of the plaque tissue, d) a device for removal of the plaque. In certain embodiments, the imaging device comprises an MRI device. In certain embodiments, the algorithm characterizes likelihood of presence of symptom-producing plaque tissue with respect to clinically-silent plaque tissue.

There is also provided herein a method for the characterizing carotid artery atherosclerotic plaque by detecting in vivo T2*-weighted imaging in carotid plaque in a subject. This method is useful to identify changes in iron content that distinguish symptomatic from asymptomatic patients with carotid atherosclerosis.

According to one embodiment, the parameter T2* is used to measure tissue magnetic susceptibility as an in vivo plaque characterization in patients with atherosclerosis.

In another broad aspect, there is provided herein a method for assessing symptomatic subjects by determining whether such subjects have lower plaque T2* values as compared with asymptomatic subjects. In certain embodiments, the symptomatic subjects had similar total iron, but less low molecular weight Fe(III) in the explanted plaques than asymptomatic patients.

In certain embodiments, the method further includes measuring one or more of calcium and copper. In certain embodiments, measurements showing a shift in iron from Fe(III) to greater amounts of T2*-shortening forms of iron; lower calcium, and greater copper in plaque, is indicative of a symptomatic subject.

In another broad aspect, there is provided herein a test which includes the in vivo measurement of intraplaque T2* using magnetic resonance imaging (MRI) to distinguish symptom-producing plaque from non-symptom-producing plaques in patients with carotid artery atherosclerosis.

In certain embodiments, the T2*-weighted imaging is quantified by using the relaxation parameter T2*, where the T2* quantification allows for an accurate estimation of plaque iron content.

In certain embodiments, the MRI images are taken of the subject's carotid artery.

In certain embodiments, the T2* measurements in having a mean value of less than or equal to 20 ms is indicative of a subject in need of treatment for carotid artery plaque removal.

In another embodiment, there is provided herein a biomarker for atherosclerotic plaque in a subject, comprising measuring, via magnetic resonance imaging, intraplaque inhomogeneity having T2* identified variable intraplaque iron content and speciation.

In certain embodiments, the speciation includes determining one or more of: the total iron, low molecular weight Fe(III), and Fe(II).

In another embodiment, there is provided herein a method for assessing patients with carotid artery related disorders that includes detecting and/or quantifying plaque T2* in such patients.

In certain embodiments, the T2* is measured over an entire plaque volume of the patient.

In another embodiment, a deceased level of paramagnetic-Fe(III) complexes is indicative of a carotid artery related disorder.

These advantages will now be illustrated by the following non-limiting examples. The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. All publications, including patents and non-patent literature, referred to in this specification are expressly incorporated by reference. The following examples are intended to illustrate certain preferred embodiments of the invention and should not be interpreted to limit the scope of the invention as defined in the claims, unless so specified.

### EXAMPLES

### Methods

Patient population. Thirty-nine patients referred for carotid endarterectomy were prospectively enrolled. Patients with ferromagnetic metal, active implants such as pacemakers, aneurysm clips, known claustrophobia, and those who were unable to provide informed consent were excluded from enrollment. No patients had hemochromatosis or significant transfusion history. Patients gave written informed consent to participate in this Institutional Review Board—approved human subjects investigation. Clinical assessment at baseline using both patient interview and chart review documented presence or absence of symptoms (symptomatic and asymptomatic patients, respectively) attributable to the carotid artery disease, such as transient ischemic attack or cerebrovascular accident in the distribution of the diseased artery and absence of other source of embolism.

Preoperative in vivo carotid magnetic resonance protocol. Carotid magnetic resonance examinations were performed using a 1.5-T scanner (Magnetom Avanto, Siemens Medical Solutions, Inc., Malvern, Pa.) and 4-channel surface radiofrequency coils placed over the neck (Machnet B V, Eelde, the Netherlands). After localization, single-shot axial steady-state free precession images were acquired using thin overlapping sections. These sections were transferred to a 3-dimensional viewer for localization of a plane demonstrating maximum carotid plaque; this slice location was then used for T2* measurement. T2*, a relaxation parameter that has been shown to be directly related to iron content in other tissues, was measured in the predetermined slice using an electrocardiography-triggered, segmented, multiple-echo, gradient-echo acquisition with echo times (TEs) of 2.7, 7.6, 12.5, 17.4, and 22.5 ms. Chemical shift selective fat suppression and double inversion recovery blood suppression were both used to improve delineation of the vessel wall. Matrix size and field of view provided in-plane spatial resolution of 0.5×0.5 mm and slice thickness was 3 mm for these acquisitions. Using the images from all 5 TEs, a region of interest was drawn encompassing the plaque and a monoexponential decay curve was fit to compute T2*.

A subset of patients also underwent T1-weighted (T1W), T2-weighted (T2W), and proton density-weighted (PDW) imaging at the same locations as T2* imaging. Typical scan parameters for these additional acquisitions were as follows. For T1W: TR 986 ms, TE 9 ms; for T2W: TR 1,978 ms, TE 79 ms; and for PDW: TR 2,030 ms, TE 9 ms. All were acquired with 3-mm slice thickness and 0.5×0.5 mm in-plane resolution. As previously done, each of these images was rated qualitatively as hypointense, isointense, or hyperintense based on signal intensity relative to skeletal muscle in the same image. T1W, T2W, PDW, and T2* image analysis were all performed blinded to patient history. Carotid T2* measurement reproducibility was confirmed by having the same T2* magnetic resonance images processed by 2 independent observers and by having a subset of patients undergo repeated T2* MRI acquisitions at adjacent slice locations.

Ex vivo plaque analysis. With explantation of the plaque at carotid endarterectomy, each patient's carotid plaque was divided into halves. One-half underwent fixation and sectioning for histopathology, including staining with hematoxylin and eosin and Prussian blue. Slides were also stained for glycophorin A and factor VIII. Histopathology slides were inspected by a single cardiovascular pathologist (P. B.), who assigned plaque stage (I to VI), Prussian blue score (0 to 4+), glycophorin A score (0 to 4+), and factor VIII score (0 to 4+) by aggregate review of all slides for each patient blinded to symptom status. The other one-half of the plaque was fresh-frozen for subsequent analysis with EPR to measure paramagnetic iron species in the plaque followed by ICP-MS to measure total iron content.

EPR spectra were recorded with a finger Dewar at 77 K with a Bruker ER 300 spectrometer (Bruker BioSciences, Billerica, Mass.) operating at X-band with 100-KHz modulation frequency and a TM₁₁₀ cavity as described previously. Tissue samples (200 to 550 mg) were cut into small pieces that were loaded into the Dewar containing liquid nitrogen and placed within the EPR spectrometer cavity. The EPR instrument parameters used were as follows: gain 5×10⁴, modulation amplitude 5 G, time constant 82 ms, scan time 131 s, microwave power 63 mW, and number of scans 10. A rhombic iron signal was seen at g≈4.3, which is characteristic of low molecular weight iron complexes. Iron levels were quantified by comparing the amplitude of the signal with standard curves generated by using known concentrations of Fe(III)-desferrioxamine (1:1 complex, generated from the addition of known concentrations of acidic FeCl₃ standard to desferrioxamine followed by titration to pH 6) under identical conditions.

The total iron content of plaque in a portion of each sample upon completion of EPR analysis was measured with ICP-MS. The samples were dried in an oven at 100° C. overnight. A portion of the sample (0.025 to 0.2 g) was placed inside a quartz vessel with 3 ml of high-purity (Fisher ACS Plus, Fisher Chemical, Pittsburgh, Pa.) nitric acid and 7 ml of deionized water for digestion and then placed in a closed trifluoromethoxyl vessel in an Ethos TC microwave digestion system (Milestone, Bergamo, Italy). The temperature was increased from 0° C. to 180° C. in the initial 10 min and then held at 180° C. an additional 10 min before the vessels were cooled and opened. Samples were diluted to 30 ml with deionized water and then placed into 30-ml low-density polyethylene plastic bottles. Samples were analyzed by either an Element 2 ICP-sector field-MS (Thermo Finnigan, Bremen, Germany) used in medium resolution (R=4,000) or a Sciex ELAN 6100DRC^{plus} (PerkinElmer, Waltham, Mass.) with methane as the reaction gas to minimize spectral overlaps so that iron could be measured at its major isotope. Calcium was measured at m/z 44 on the Element 2 and m/z 40 on the ELAN DRC^{plus}. Copper was measured at m/z 63 and 65 on the Element 2 and m/z 65 on the ELAN DRC^{plus}. Cobalt (100 ppb) was added to each sample and standard and used as an internal standard to correct for instrument drift and changes in sensitivity due to high, variable calcium concentrations. The samples were introduced into the ICP-MS by a PFA-ST concentric nebulizer (Elemental Scientific, Omaha, Nebr.) and a PFA spray chamber (Elemental Scientific). The sample was pumped at an uptake rate of 0.5 ml/min to the nebulizer.

Statistical analysis. Continuous data are expressed as mean±SE. The relationships between continuous variables were examined by nonparametric Spearman correlation. A nonparametric exact Wilcoxon rank sum test was used to compare symptomatic to asymptomatic patients with respect to each continuous variable. A value of p<0.05 was regarded as statistically significant. Comparison of frequencies of patient characteristics was done using the Fisher exact test.

Results

Of the 39 study subjects, 19 were men (Table 2); all women in this study group were postmenopausal. Eleven subjects had symptoms attributable to their carotid artery disease: 6 strokes and 5 transient ischemic attacks occurred in the distribution of the carotid lesions. A total of 28 asymptomatic subjects had high-grade carotid artery stenosis identified by ultrasonography that was prompted in most cases by auscultatory findings on physical examination. Confirmation of stenosis severity to proceed with endarterectomy was provided in all subjects by either invasive angiography (23 subjects) or contrast-enhanced magnetic resonance angiography. Symptomatic and asymptomatic patient groups did not significantly differ in age (64.9±2.6 years vs. 66.5±1.2 years, p=0.70), frequency of diabetes (6 of 11 patients vs. 10 of 28 patients, p=0.47), or male gender (4 of 11 patients vs. 15 of 28 patients, p=0.48).

TABLE 2

| | |
|---|---|
| Age, yrs | 66.0 ± 7.0 |
| Male gender (%) | 19 (49) |
| Diabetes, n (%) | 16 (42) |
| Current or former smoker, n (%) | 26 (72) |
| Symptomatic due to carotid lesion, n (%) | 11 (28) |
| Serum hemoglobin, mg/dl | 12.6 ± 2.3 |

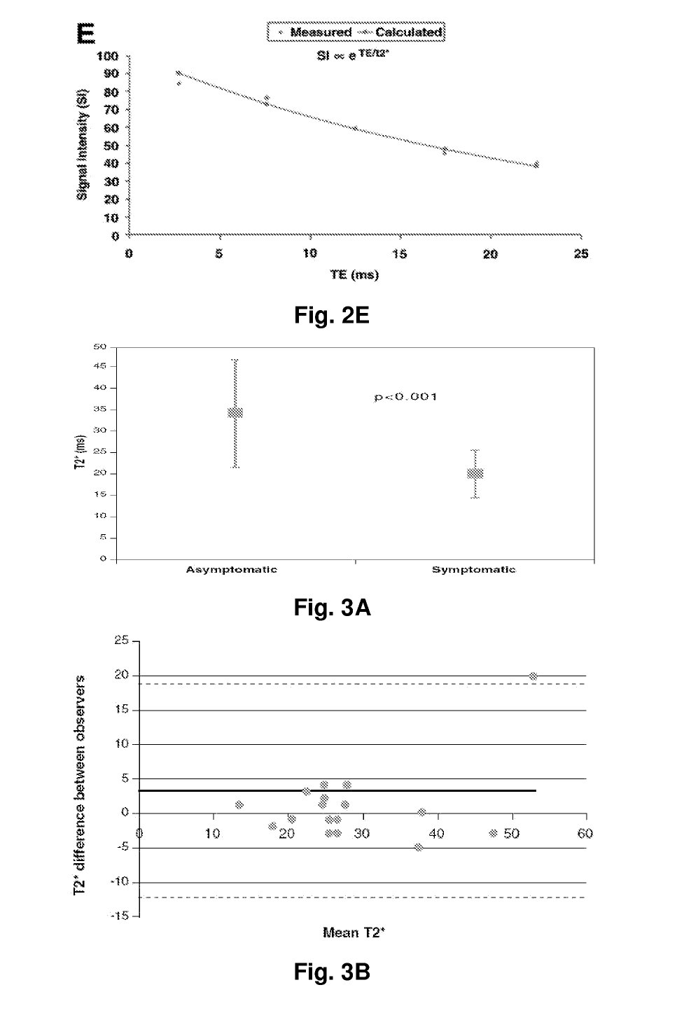

Four subjects, all asymptomatic, could not undergo MRI because of severe claustrophobia. Early in the study, the MRI T2* acquisition in 3 subjects who had difficulty lying still, 1 symptomatic, resulted in severe motion artifact and uninterpretable MRI data. Subsequent use of an inflatable neck cushion that fixed the neck and coils in place during imaging eliminated motion artifacts. Average time for carotid T2* data acquisition was 1 min per slice. Sample magnetic resonance acquisitions are shown in FIG. 2.

Plaque T2* measurements were significantly shorter, indicating greater levels of T2*-shortening iron, in plaques from symptomatic versus asymptomatic patients (mean 20.0 ms vs. 34.4 ms, respectively, p<0.001) (FIG. 3A). Repeated measurements showed good interobserver agreement (r=0.88) of plaque T2* quantification (FIG. 3B). T2* measurements compared in 14 pairs of adjacent 3-mm image slices showed good reproducibility: the difference between T2* values in adjacent slices averaged 6.5±4.4 ms. Additional T1W, T2W, and PDW imaging was performed in 11 subjects; their multispectral analyses are summarized in Table 3.

TABLE 3

| Sub- | | | | | | |
|---|---|---|---|---|---|---|
| ject | Symptom | MRI | MRI | MRI | MRI | Intraplaque |
| ID | Status* | T1W† | T2W† | PDW† | T2*, ms | Hemorrhage‡ |
| 16 | S | 0 | 0 | − | 14 | Y |
| 25 | A | + | + | + | 35 | Y |
| 32 | A | 0 | + | + | 27 | N |
| 33 | A | − | + | − | 24 | Y |
| 42 | A | + | + | + | 28 | N |
| 43 | A | 0 | + | 0 | 25 | N |
| 46 | A | 0 | − | 0 | 38 | N |
| 48 | A | 0 | 0 | + | 34 | N |
| 50 | A | U | + | U | 32 | N |
| 51 | S | − | − | − | 19 | Y |

*Symptom status (A = asymptomatic; S = symptomatic) determined by clinical history and chart review.
†Hypointense (−), isointense (0), hyperintense (+), or uninterpretable (U) for plaque signal intensity on T1-weighted (T1W), T2-weighted (T2W), and proton density-weighted (PDW) magnetic resonance imaging (MRI).
‡Intraplaque hemorrhage defined by histopathology: Y = present; N = absent.

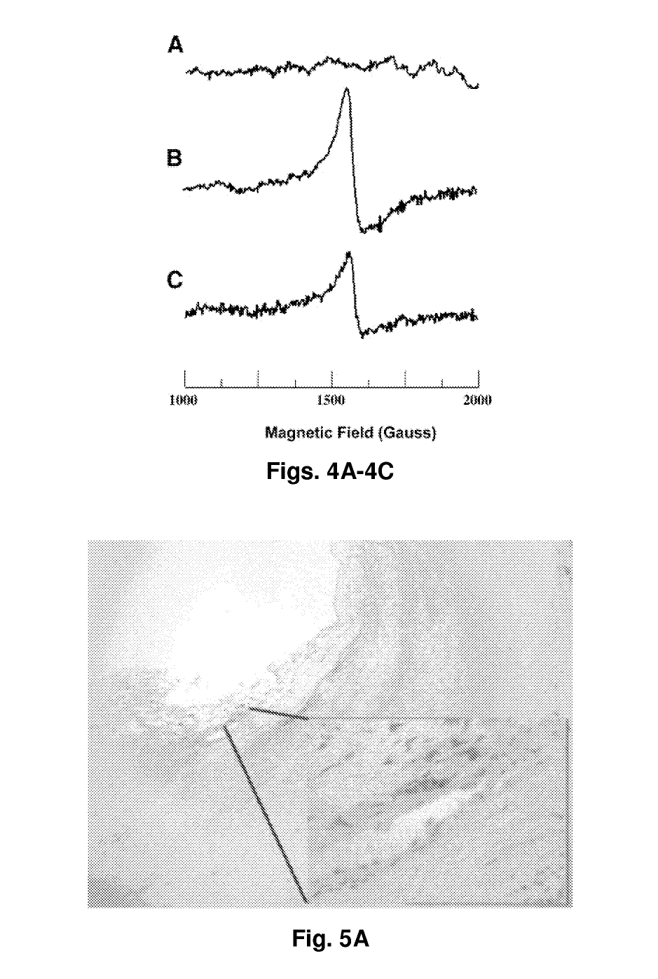

EPR-detectable Fe(III) was present in all but 4 patient plaques (3 symptomatic, 1 asymptomatic; p=NS) as a strong rhombic Fe(III) signal with a g value of 4.3, centered at 1,500 Gauss (FIG. 4). This signal has been previously assigned to low molecular weight Fe(III)-complexes. Quantitative results from in vivo T2* measurements, EPR iron content, and total iron, calcium, and copper content by ICP-MS are summarized in Table 4.

TABLE 4

| | Symptomatic | Asymptomatic | p |
|---|---|---|---|
| Variable | Patients | Patients | Value |
| T2*, ms | 20.0 ± 1.8 | 34.4 ± 2.7 | 0.0006 |
| EPR Fe(III), μg/g | 7.3 ± 3.8 | 17.7 ± 4.0 | 0.025 |
| ICP-MS total iron, μg/g | 138.6 ± 36.5 | 165.8 ± 48.3 | NS |
| ICP-MS copper, μg/g | 3.2 ± 0.5 | 1.7 ± 0.1 | 0.004 |
| ICP-MS calcium, mg/g | 37.5 ± 10.8 | 123.6 ± 19.3 | 0.003 |

Both symptomatic patients and asymptomatic patients had similar levels of total iron by ICP-MS (mean 90.5 vs. 72.8 μg/g, respectively). Whereas total iron content by ICP-MS was similar, levels of paramagnetic Fe(III) complexes by EPR were significantly lower in plaques from symptomatic versus asymptomatic patients (mean 7.3 vs. 17.7 μg Fe(III)/g tissue wet weight, p=0.025). There was significantly less calcium and more copper by ICP-MS in symptom-producing versus non-symptom-producing plaques.

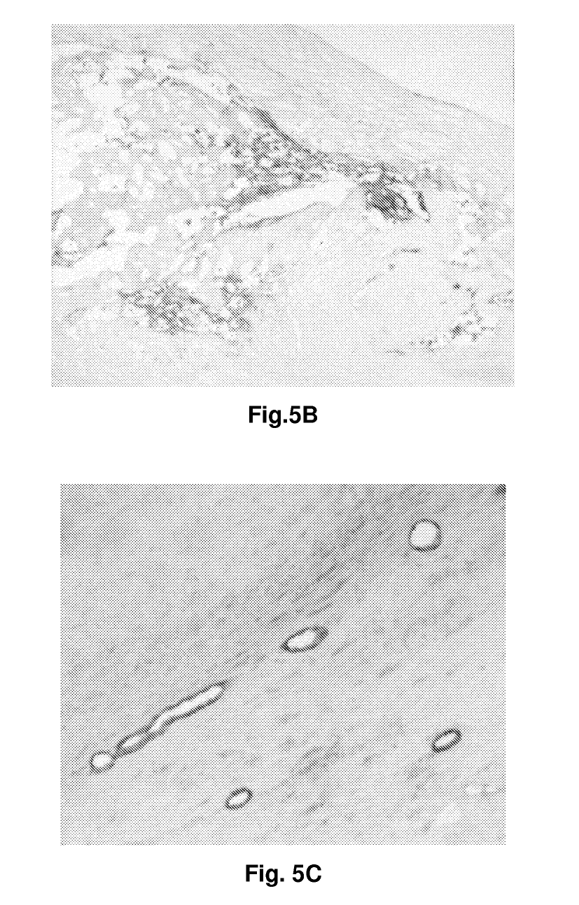

Overall, the majority of plaques demonstrated advanced features (Stary class IV to VI). Histopathology demonstrated both red blood cell degradation as well as plaque disruption with intraplaque hemorrhage, both of which may be sources of intraplaque iron (FIG. 5). A positive correlation was found between glycophorin A and Prussian blue (p=0.05), as well as between glycophorin A and Factor VIII (p=0.001).

Discussion of Example

In patients with carotid artery atherosclerosis, in vivo MRI measurement of intraplaque magnetic inhomogeneity with T2* identified variable intraplaque iron content and speciation; this quantitative, reproducible parameter distinguished symptomatic from asymptomatic patients.

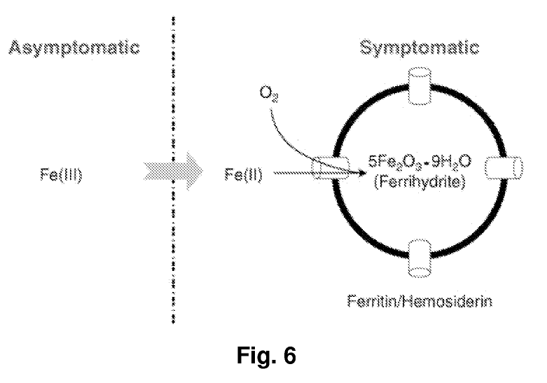

With ex vivo analyses, total iron was similar in both groups, but low molecular weight Fe(III) was lower in symptom-producing plaques; conversion to other forms of iron may produce this shortening of T2* relaxation time (FIG. 6).

Iron catalyzes free radical production which is a key step for lipid peroxidation and atherosclerosis development. Both ferritin and hemosiderin are known to significantly shorten T2* relaxation time (29); in fact, T2* may be considered a “biomarker” of iron aggregation. Both iron and copper have been implicated in previous studies of metal ions in atherosclerosis development, although iron affects T2* to a much greater extent, because its magnetic moment is larger and its quantity in plaque is nearly 2 orders of magnitude greater than that of copper. Since similar levels of total iron were present in both groups, these results are consistent with a shift of iron from low molecular weight paramagnetic Fe(III) complexes, and possibly from EPR-invisible diamagnetic Fe(II) complexes, to other species such as hemosiderin and ferritin, which are known to markedly shorten the T2* relaxation time.

The majority of previous work in humans to address the role of iron in atherosclerosis has lacked direct examination of human atherosclerotic plaques for iron, making it difficult to lend credence to the iron hypothesis or make a direct histological link to plaque composition itself. While systemic factors may contribute to a milieu that favors atherosclerosis, plaque development occurs in discrete locations, mandating direct studies of plaque's microenvironment to gain insight into molecular mechanisms.

In symptomatic patients, a shift of the type of iron complexes present seemed to occur with shortening of T2*. This shift may be secondary to an increase in ferritin-bound iron, which is consistent with previous studies documenting increased levels of ferritin in atherosclerotic plaque and a positive correlation between ferritin levels and apoptotic cell death.

It is notable that iron in plaques on histopathology occurred both at sites of intraplaque hemorrhage, an established marker of plaque instability, as well as in macrophages. The correlation of clinical manifestations with explanted iron quantification supports the notion that iron does play a role in the natural history of atherosclerosis.

Previous studies have implemented conventional T1W, T2W, proton density, and time-of-flight imaging with good ability to classify plaques, particularly those with intraplaque hemorrhage, based on multicontrast analysis. The limited number of patients undergoing multicontrast MRI with T2* quantification precludes direct comparison, especially in light of variable plaque classification schemes that are based on subjective assessment of multicontrast magnetic resonance images, even among senior investigators (34,35).

However, given: 1) its greater sensitivity to and specificity for intraplaque iron; 2) its clinical relevance in distinguishing symptom-producing plaques; and 3) its quantitative nature that does not require qualitative assessment of relative signal intensity, plaque T2* quantification as developed in this work should be a useful addition to the assessment of patients with carotid artery disease. Current decision-making regarding carotid endarterectomy relies on patient history and percent stenosis, despite a stroke rate of 15% to 20% in asymptomatic patients with 50% to 69% stenosis that do not undergo revascularization. The prognostic value of multispectral qualitative plaque MRI and intraplaque T2* measurement would be best evaluated in a prospective study, as we have ongoing at our institution. This approach may help identify asymptomatic patients with “vulnerable plaque” that would benefit from interventions to reduce the stroke rate in this population.

It is to be understood, that in other embodiments, use of susceptometry techniques can be used to measure tissue iron.

Due to the 2-dimensional nature of the T2* acquisition, only one slice was selected for plaque T2* measurement. Although the region of interest for T2* measurement was drawn such that it encompassed the entirety of the plaque at a slice showing maximum plaque, this region might not necessarily reflect the T2* of the entire plaque. It is also to be understood that a volumetric assessment can be achieved by using a 3-dimensional T2* acquisition technique. The present Example demonstrates predictive value using single-slice in vivo plaque T2* compared with ex vivo EPR and ICP-MS measurements of larger plaque sections supporting further work to measure T2* over an entire plaque volume.

The results did indicate reproducibility of T2* plaque measurement in patients undergoing repeated T2* imaging at 1 setting as well as off-line quantification of T2* images by multiple independent observers.

Much attention has focused on detecting atherosclerosis with calcium screening. The inventors herein found that carotid plaques in symptomatic patients actually had lower calcium content, which is consistent with previous work using B-mode ultrasound demonstrating lower calcium content in carotid plaques from symptomatic versus asymptomatic patients. In addition, histopathology and intravascular ultrasound of the coronary arteries have demonstrated that lesions associated with chronic stable angina are more extensively calcified than those associated with acute coronary syndromes. In the current Example, there was concern that calcium would interfere with the measurement of T2*, because calcium produces a very low signal intensity on T2*-weighted gradient echo imaging, potentially shortening the T2* relaxation time. Ex vivo analysis demonstrated lower calcium values in patients with shorter T2* times, indicating that the change could not be due to calcium but another substance, namely iron.

Noninvasive carotid plaque T2* measurement distinguished plaques that produce symptoms from those in asymptomatic patients undergoing carotid endarterectomy.

The results indicate the presence of decreased levels of paramagnetic-Fe(III) complexes and similar total iron levels. With T2*-shortening, these results show a shift to aggregate iron complexes that have greater local effects on magnetic susceptibility.

It is further to be understood that the present invention, in certain embodiments, can include one or more computer-implemented processes and apparatuses for practicing those processes. Certain embodiments may also be embodied in the form of computer program code containing instructions embodied in any suitable computer-readable storage medium. For example, when the computer program code can be loaded into and executed by a computer, the computer becomes an apparatus for practicing the invention. It should also be understood that certain embodiments can also be in the form of a computer program code; for example, whether stored in a storage medium, loaded into and/or executed by a computer, or transmitted over some transmission medium, such as electrical wiring or cabling, through fiber optics, or via electromagnetic radiation, wherein, when the computer program code is loaded into and executed by a computer, the computer becomes an apparatus for practicing the invention. In another example, when implemented on a general-purpose microprocessor, the computer program code segments configure the microprocessor to create specific logic circuits.

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof. Therefore, it is intended that the invention not be limited to the particular embodiment disclosed herein contemplated for carrying out this invention, but that the invention will include all embodiments falling within the scope of the claims.

1. A method for in vivo atherosclerotic plaque characterization in a subject in need thereof, comprising:
detecting a level of one or more iron complexes in plaque in the subject, and
comparing detected level to a known value determined from subjects without an atherosclerotic condition.

2. The method of claim 1, comprising measuring carotid plaque using a T2*-weighted imaging to distinguish symptom-producing plaque from clinically-silent plaque in the subject.

3. The method of claim 2, comprising distinguishing magnetic susceptibility to symptom-producing plaque from clinically-silent plaque in the subject.

4. The method of claim 2, wherein the iron complex comprises paramagnetic-Fe(III) complexes.

5. A method of claim 4, wherein the presence of decreased levels of paramagnetic-Fe(III) complexes is indicative of symptom-producing plaque.

6. The method of claim 2, wherein greater concentrations of one or more iron complexes are present atherosclerotic plaque compared with normal arterial tissue.

7. The method of claim 2, wherein change in iron concentration is qualitatively measured using magnetic resonance T2*-weighted imaging.

8. The method of claim 2, wherein change in iron concentration is quantified using a T2* relaxation parameter.

9. The method of claim 8, wherein T2* quantification allows for a substantially accurate estimation of tissue iron content in the plaque.

10. The method of claim 9, using MRI imaging of the plaque in vivo.

11. A method for detecting an atherosclerotic condition in a subject, comprising:
a) acquiring MRI data from the subject with a magnetic resonance imaging system that measures T2* in plaque present in the subject; and
b) comparing the MRI data of step a) to one or more known parameters;
c) indicating a difference between the data of step a) with the known parameters.

12. The method of claim 11, comprising:
obtaining at least a first set of image data created in response to an MRI scan of at least a portion of plaque in the subject, wherein the MRI scan includes T2* weighted imaging; and
determining the presence of at least one iron complex present in the plaque;
wherein the presence of the at least one iron complex at an altered level is indicative of symptom-producing plaque in the subject.

13. The method of claim 12, wherein the subject being scanned was injected with a contrast agent.

14. A system for plaque characterization, comprising:
an imaging system generating at least a first set of image data of plaque in a subject in response to a first magnetic energy level; the first set of image data corresponding to a T2*-weighted imaging of the plaque, and
a processing device in communication with the imaging system obtaining at least the first set of image data from the imaging system and displaying an indication of one or more iron complexes present in the plaque.

15. The system of claim 14, wherein the imaging system comprises a magnetic resonance imaging system.

16. The system of claim 15, wherein the imaging system is remotely located from the processing device.

17. The system of claim 16, wherein the processing device is in communication with the imaging system over a network.

18. A computer program product for plaque characterization in cardiac applications, the product comprising: a storage medium readable by a processing circuit and storing instructions for executing a method for plaque characterization by the processing circuit, the method comprising a method as described in any of the preceding claims.

19. A method to improve risk assessment for an acute cardiovascular event in a patient presenting with plaque, comprising
detecting a level of one or more iron complexes in plaque in the subject, and
comparing the detected level to a known value determined from subjects without an atherosclerotic condition.

20. The method of claim 19, wherein the level of the iron complex is detected with a magnetic resonance imaging scan.

21. A method for characterizing response to therapy in a clinical trial of a medication, device and/or drug comprising determining a level of one or more iron complexes in a trial participant.

22. An imaging system for differentiating between symptom-producing plaque and clinically-silent plaque tissue in a subject, comprising:
a) at least one imaging device for providing imaging data;
b) a processor receiving the imaging data and executing software to evaluate the image data according to at least one algorithm; and
c) the algorithm processing the imaging data and outputting information relating to at least a level of one or more iron complexes present in the plaque tissue.

23. The imaging system of claim 22, further including, for guided removal of the plaque tissue, d) a device for removal of the plaque.

24. The system of claim 22, wherein the imaging device comprises an MRI device.

25. The method of claim 22, wherein the algorithm characterizes likelihood of presence of symptom-producing plaque tissue with respect to clinically-silent plaque tissue.
